(19) United States
(12) Patent Application Publication
(10) Pub. No.: US 2011/0060245 A1
(43) Pub. Date: Mar. 10, 2011

(54) DEVICE FOR MEASURING INTRACRANIAL PRESSURE IN NEWBORNS AND BABIES AND A SUPPORTING MEMBER FOR SAID DEVICE

(76) Inventors: Gennadiy Konstantinovich Piletskiy, Ryazan (RU); Nikolai Gennadyevich Piletskiy, Ryazan (RU)

(21) Appl. No.: 12/736,773

(22) PCT Filed: Mar. 27, 2009
(86) PCT No.: PCT/RU2009/000145

§ 371 Date: Nov. 8, 2010

(30) Foreign Application Priority Data
May 6, 2008 (RU) 2008117585

(51) Int. Cl. A61B 5/03

(52) U.S. Cl. 600/561

(57) ABSTRACT

The invention relates to medicine. The inventive device for measuring intracranial pressure in newborns and babies comprises a body, a supporting member with a leveling surface on the end face thereof, a movable bush which is provided with the supporting member coaxially attached thereto and which bush is arranged in the body so as to be capable of carrying out a limited reciprocating motion to produce a static load on the bregmatic fontanel, a rod with a flat base which is accommodated in the movable bush cavity so as to be capable of moving with respect to the supporting member and producing an impact action for deforming the bregmatic fontanel, a rod moving system provided with a measuring coil which is connected to a control, processing and display unit for converting a signal received from the measuring coil in order to determine the intracranial pressure and display the obtained results. The supporting member is designed in the form of a bush with a leveling surface on the end face and is provided on the outer side with a limiter of the deformation produced by the static load on the bregmatic fontanel, the leveling surface of the supporting member is projected beyond the contact surface of the limiter, wherein said contact surface is inclined to a plane which is tangent to the leveling surface in such a way that a distance between the contact surface and the tangent plane increases in the direction towards the periphery of the limiter of the deformation produced by the static load on the bregmatic fontanel.

### FIELD OF THE INVENTION

The invention relates to portable medial devices for pediatrics designed for functional diagnostics of the condition of cerebral hemo- and liquorodynamics, and may be used in clinical practice for noninvasive painless, fast, and accurate measurement of intracranial pressure (ICP), without the fear of infection, in early-age children by using short-duration dynamic deformation of the bregmatic fontanel during examination of sick children for cerebral diseases, and monitoring their treatment at hospital and at home.

### BACKGROUND OF THE INVENTION

There is a great number of devices for investigating ICP indirectly today. In babies, for example, such diagnostics is carried out by ultrasonic investigation of the cranium through the bregmatic fontanel. In older children, it is performed by computer tomography and magnetic resonance tomography (MRT). The devices used for this purpose do not produce an objective evaluation of the ICP, are complex, very expensive, and impossible to use by parents for repeated ICP monitoring at home.

In these conditions, it is highly important to develop devices for fast, noninvasive measurement of ICP that can be used safely and, in need, repeatedly to measure this parameter by the children's parents themselves.

The prior art devices that are used to determine ICP are based on measurement of the extent of diaphragm displacement (RU Patent No. 2,163,090) or on evaluation of changes in the magnetic impedance of the brain (U.S. Pat. No. 4,690,149 and No. 4,819,648), and ultrasonic ICP measuring devices (U.S. Pat. No. 5,388,583 and No. 5,411,028). These prior art devices have a complex design, are expensive, and can be used at hospital only.

Another prior art device for measuring ICP has a sensing component comprising a rigid ring with a flexible membrane placed in the opening thereof and adapted to be positioned on the bregmatic fontanel of a child with the use of straps, the rigid ring and membrane being secured in position to form a cavity for a fluid in which a pressure sensor is immersed (U.S. Pat. No. 4,995,401, IPC A 61B 5/00, published in 1991). Deformation of the skin covering the fontanel in this device causes the flexible membrane to be deformed accordingly and, in this way, fluid pressure in the cavity to be measured.

The straps complicate and draw out the ICP measurement procedure by this prior art device.

A further prior art device for measuring ICP through the fontanel of a child comprises a rigid component to position a pressure measuring unit with a membrane, the rigid component being held in place by an adhesive (DE Patent No. 3,928,554, IPC A 61B 5/03, published in 1991).

The difficulty of attachment, centering, and adhesive application draw out and complicate the ICP measurement procedure, making it painful in repeated ICP measurements.

The device closest to the present invention for measuring intracranial pressure in newborns and babies comprises a body, a support having two wedge-shaped projections, with an annular projection positioned between them and having a leveling area at a height of 3 to 5 mm from the base of the projections; a movable bush to secure the support therein that is disposed inside the body for limited reciprocating movement therein to apply a static load to the bregmatic fontanel; a rod having a flat base that is received within the bush cavity for movement relative to the support and producing impacts to deform the bregmatic fontanel; a mechanical system for moving the rod comprising at least one measuring winding connected to a control, processing, and display unit for converting a signal received from the measuring winding in order to determine the intracranial pressure and displaying the results obtained, the wedge-shaped projections being spaced identically at a distance of 7 to 10 mm from the rod movement axis and their supporting surfaces being below the leveling surface of the annular projection (RU Patent No. 2,303,946, IPC A 61B 3/16, published on Aug. 10, 2007).

This prior art device does not allow ICP to be measured with sufficient speed and measurement accuracy on fontanels of flat and concave shape because of the supporting surfaces of the wedge-shaped projections being located significantly below the leveling surface of the annular projection. For this reason, the device cannot be used in clinical practice on a wide scale.

None of the prior art noninvasive devices, therefore, produces the accuracy, speed, painfulness, and convenience acceptable for use in clinical practice. The need still exists for an inexpensive noninvasive ICP measuring device simple in design and convenient in use, that gives sufficient accuracy and allows numerous repeated ICP measurements to be made on fontanels of different shapes.

The closest prior art of the present invention is a supporting member for devices used to measure intracranial pressure in newborns and babies that is designed as a bush having a bore for the rod of the ICP measuring device to move therein, apertures for releasing the air during the movement thereof, and a leveling surface at the end thereof, said leveling surface being provided between two wedge-shaped projections having supporting surfaces below said leveling surface (RU Patent No. 2,303,946, IPC A 61/B 3/16, published on Aug. 10, 2007).

Location of the supporting surfaces of the wedge-shaped projections significantly below the leveling surface of the annular projection does not guarantee a high accuracy of measurement on fontanels of flat and concave shape, or safety in use.

### SUMMARY OF THE INVENTION

The invention is aimed at improving the operating properties of a device for measuring ICP through the bregmatic fontanel in newborns and babies.

The technical result of the present device for measuring intracranial pressure in newborns and babies consists in raising the accuracy of ICP measurement on fontanels of different shapes, including concave, flat, and convex shapes; reducing the time needed for the measurement as a whole by a factor of several times; simplifying the measurement procedure and making it comfortable; and allowing ICP to be measured at hospital and at home by the children's parents themselves.

The technical result of the present supporting member for a device for measuring intracranial pressure in newborns and babies consists in that it allows the accuracy of ICP measurement to be improved, is simple in design, convenient, and safe when applied to fontanels of different shapes, including concave, flat, and convex shapes.

The above technical result is achieved in a device for measuring intracranial pressure in newborns and babies comprising a body; a supporting member having a leveling surface at the end thereof; a movable bush having said supporting member secured coaxially thereon and disposed within the body for limited reciprocation to produce a static load on the bregmatic fontanel; a rod having a flat base and disposed within the movable bush cavity for moving relative to the supporting member and producing an impact force to deform the bregmatic fontanel; a rod moving system having at least one measuring winding connected to a control, processing, and display unit converting a signal received from the measuring winding in order to determine the intracranial pressure and display the results obtained, wherein the supporting member according to the invention has on the outer surface thereof a limiter of bregmatic fontanel deformation by static load; the leveling surface of the supporting member extending beyond the contact surface of said limiter, said contact surface being inclined toward a plane normal to the axis of the movable bush and tangent to the leveling surface of the supporting member with an increase in the distance between said surface and said plane in the direction toward the periphery of the limiter of bregmatic fontanel deformation by static load.

It is preferred to have the limiter of bregmatic fontanel deformation by static load in the form of at least one projection on the side surface of the supporting member, the inclination angle of the contact surface of said supporting member to said plane being equal to between 5° and 15°.

It is preferred to have the distance from the plane tangent to the leveling surface to a point furthest therefrom on the contact surface of the limiter of bregmatic fontanel deformation by static load within 0.5 to 1.0 mm.

It is preferred to make the limiter of bregmatic fontanel deformation by static load in the form of a pair of symmetrical petals each having a contact surface merging smoothly with the leveling surface of the supporting member.

It is preferred for the limiter of fontanel deformation by static load to have each of the petals opening at an angle of 70° to 85°, a length between 2 and 3 mm along the common axis of symmetry of the petals, and a width of 4 to 6 mm.

It is preferred to have a movable bush and supporting member provided with respective cavities and apertures for releasing air during rod movement.

It is preferred to provide the supporting member with side viewing windows in the form of U-shaped depressions or apertures located symmetrically between the petals.

It is preferred to make the leveling surface of the supporting member in the shape of a continuous or split ring and rounded in the axial section thereof.

It is preferred to provide the rod with a flat base having an area of 0.2 to 1.5 mm².

It is preferred to make the supporting member in the form of a removable bush.

It is preferred to have an electromagnetic system for moving the rod, which comprises a permanent magnet fitted on the rod; and a measuring winding disposed in the movable bush and positioned with respect to the permanent magnet so as to control the direction and speed of rod movement.

It is preferred to have a control, processing, and display unit capable of producing voltage of opposite polarities across the measuring coil for retracting the rod to the initial position thereof and generating an impulse to propel the rod in the direction toward the supporting member.

It is preferred to have a contactless indicator of a reference static load applied to the bregmatic fontanel for measurement purposes, said indicator being designed as a sensor indicating the position of the bush relative to the body.

It is preferred to provide the sensor indicating the position of the movable bush relative to the body with a generator, an additional bush having a cavity, and a coil arranged on the movable bush and body, respectively, to alter the coil winding inductance during movable bush-movement, the coil winding being connected to the generator circuit.

It is preferred to connect the generator of the sensor indicating the position of the movable bush relative to the body to the control, processing, and measurement data display unit.

The technical result is also achieved by providing the supporting member for the device for measuring the intracranial pressure in newborns and babies in the form of a bush having a leveling surface at the end, according to the invention, on the outer surface thereof with a limiter of bregmatic fontanel deformation by static load, the leveling surface of said limiter extending beyond the contact surface of said limiter, the contact surface being inclined to a plane tangent to the leveling surface, with the distance from the leveling surface to said plane increasing toward the periphery of the limiter of bregmatic fontanel deformation by static load.

It is preferred for the limiter of bregmatic fontanel deformation by static load in the supporting member to be designed as at least one projection on the side surface of the supporting member, said projection having a contact surface extending at an inclination angle of 5° to 15° to said plane tangent to the leveling surface.

It is preferred that, in said supporting member, the distance from said plane tangent to the leveling surface of the supporting member to a point furthest therefrom on the contact surface of the limiter of bregmatic fontanel deformation by static load ranges between 0.5 and 1.0 mm.

It is preferred to make the limiter of bregmatic fontanel deformation by static load in the form of a pair of symmetrical petals, each having a contact surface merging smoothly with the leveling surface.

Each petal of the limiter of bregmatic fontanel deformation by static load preferably has an opening angle of 70° to 85°, a length of 2 to 3 mm along the common axis of symmetry of the petals, and a width of 4 to 6 mm.

It is preferred to provide the supporting member with side viewing windows in the form of U-shaped depressions or apertures disposed opposite one another between the petals.

It is preferred to have the leveling surface of the supporting member rounded in the axial section thereof and made in the shape of a continuous or split ring.

It is preferred to provide the supporting member with a bore for the rod of the device for measuring intracranial pressure to move therein and apertures for releasing air during the movement of said rod.

It is the idea of the invention to use a limiter of bregmatic fontanel deformation by static load in order to improve the accuracy of ICP measurement, the use of an electromagnetic system to control the rod movement making the device fast and simple in application.

The patent search has not uncovered any devices similar to the present device, and the present device, therefore, complies with the “novelty criterion.” The idea of the invention does not follow obviously from prior art inventions, so the present invention complies with the “inventive level” criterion.

### BRIEF DESCRIPTION OF THE DRAWINGS

FIG. 1 is a cutaway view of the device for measuring ICP.

FIG. 2 is a view of the supporting member with a limiter of bregmatic fontanel deformation by static load as viewed from the contact surface (bottom view).

FIG. 3 is a cutaway view of an embodiment of the supporting member having a viewing window and leveling surface in the form of a split ring.

FIG. 4 is a cutaway view of another embodiment of the supporting member having a viewing window and leveling surface in the form of a continuous ring.

FIG. 5 is a view of the rod position within the supporting member prior to ICP measurement.

FIG. 6 is a view of the rod extended to a maximum distance beyond the leveling surface of the supporting member.

FIG. 7 is a diagrammatic view of the principal components of the ICP measuring device illustrating the operation thereof.

FIG. 8 is a view of the device positioned on a child's bregmatic fontanel at the ICP measurement time.

### DESCRIPTION OF AN EMBODIMENT OF THE INVENTION

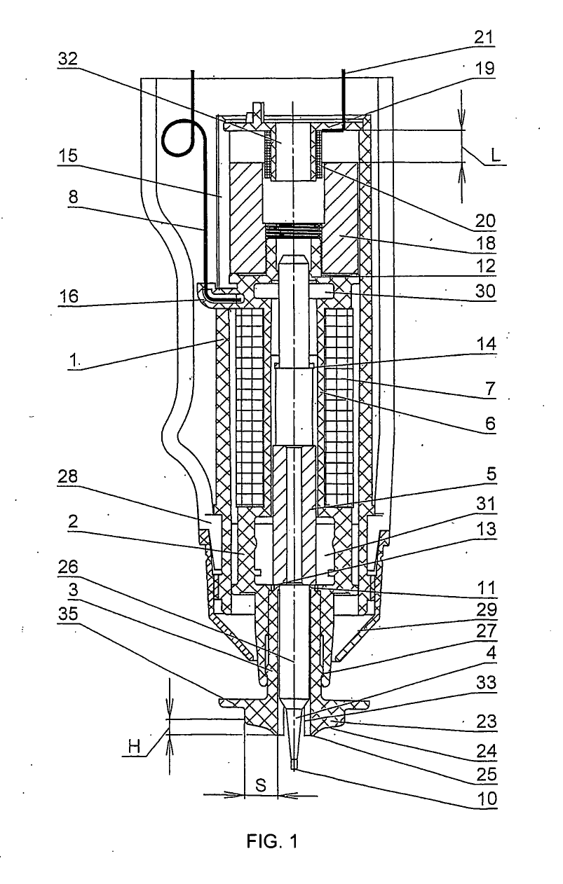

The ICP measuring device (FIG. 1) comprises a body 1 made of plastics and a movable bush 2 having a supporting member 3 in the form of an end piece disposed within body 1 for limited reciprocation therein to apply static load to a child's bregmatic fontanel. A rod 4 of a nonmagnetic material is disposed in the cavity of movable bush 2 for movement relative to supporting member 3 for deforming the fontanel by a short-duration impact force.

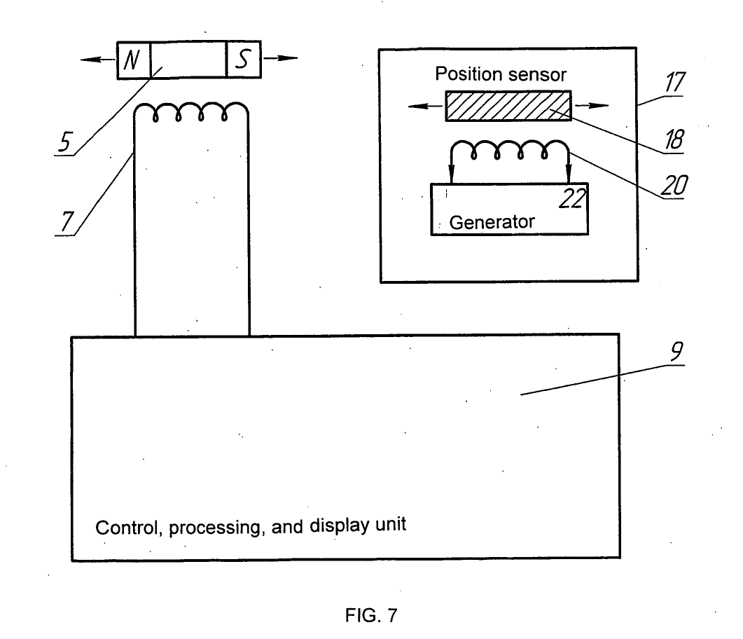

Rod 4 of the device is reciprocated by an electromagnetic system that comprises a permanent magnet 5 positioned on rod 4 and a coil 6 placed in the wall of movable bush 2 and having at least one measuring winding 7 disposed relative to permanent magnet 5 to control the direction and speed of movement of rod 4. Measuring winding 7 is connected by a lead 8 to a control, processing, and display unit 9 (FIG. 7).

Rod 4 is made of plastics and has a flat base 10 measuring 0.2 to 1.5 mm² in area. An area smaller than 0.2 mm² is likely to produce a sensation of pain during the measurement procedure, while an area larger than 1.5 mm² does not guarantee skin compression in the area of the bregmatic fontanel. Supporting member 3 is a bush having a bore (FIG. 3) of a diameter (G) between 3.0 and 3.5 mm for rod 4 to move therein.

A lower and an upper sliding bearings 11 and 12, respectively, formed in movable bush 2 allow rod 4 to move freely longitudinally within coil 6 and supporting member 3. The downward movement of rod 4 is limited by surface 13 of lower sliding bearing 11. Movement of rod 4 to its topmost initial position within movable bush 2 is limited by surface 14 of the projection of rod 4 and top sliding bearing 12.

Body 1 has a longitudinal slot 15 to receive a mechanical limiter 16 made integrally with movable bush 2 to limit vertical reciprocation thereof and prevent rotation thereof relative to body 1. A lead 8, a length of which is built into the cavity of mechanical limiter 16, is used for connecting measuring winding 7 to control, processing, and display unit 9.

The ICP measuring device is provided with a contactless indicator of a reference static load applied to the fontanel, said indicator being designed, for example, as a sensor 17 (FIG. 7) indicating the position of movable bush 2 relative to body 1. Said sensor 17 comprises an additional bush 18 and a fixed coil 19 provided on movable bush 2 and body 1, respectively, a winding 20 of fixed coil 19 being connected by a lead 21 to the circuit of a generator 22 connected to control, processing, and display unit 9 (FIG. 7).

The static load (made up by the weight of movable bush 2 and supporting member 3, measuring winding 7 and additional bush 18, and the weight of rod 4 and permanent magnet 5) needed for measurement purposes has to be between 20 and 30 grams. A weight under 20 grams does not provide the required stabilization of movable bush 2 and supporting member 3, while a weight over 30 grams causes a significant increase in the tonometric intracranial pressure because of extra deformation of the fontanel.

Supporting member 3 (FIGS. 1, 2, and 3) has on the outer surface thereof a limiter 23 of bregmatic fontanel deformation by static load that has a contact surface 24, for example, a surface of revolution, and a leveling surface 25. In turn, it is preferred to make supporting member 3 in the form of a removable bush having a bore for rod 4 to move therein, said bush serving as the end piece of the device for measuring intracranial pressure.

Limiter 23 of fontanel deformation by static load is positioned on the side wall of supporting member 3, between movable bush 2 and leveling surface 25, and can increase the area of contact between the fontanel and contact surface 24 when ICP decreases. For this purpose, leveling surface 25 of supporting member 3 extends beyond contact surface 24 of said limiter 23, said contact surface being inclined to a plane normal to the axis of movable bush 2 and tangent to leveling surface 25 of the supporting member with an increase in the distance from contact surface 24 to said plane in the direction toward the periphery of said limiter 23.

In this position, the inclination angle (a) (FIG. 3) of contact surface 24 to said plane is varied preferably between 5° and 15°.

Supporting member 3 may have several embodiments, including one having leveling surface 25 rounded off in axial section and having the shape of a split ring (FIG. 3) or continuous ring (FIG. 4).

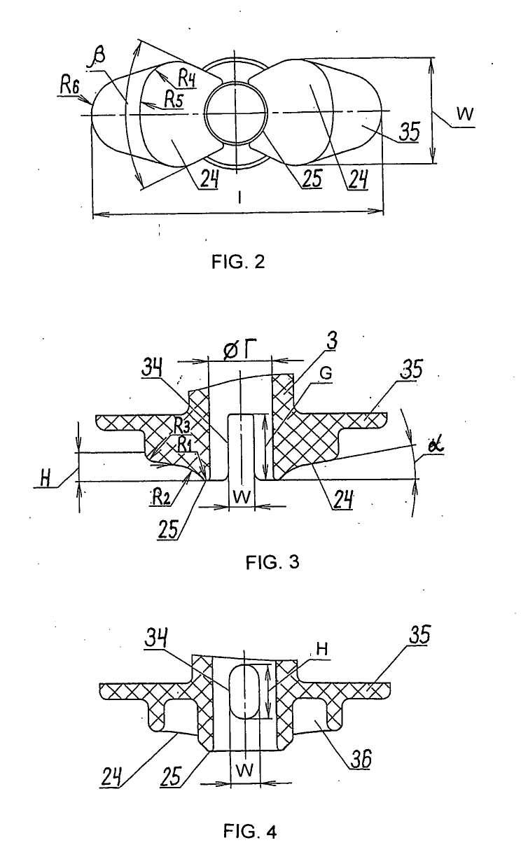

In a preferred embodiment (FIG. 2), said limiter 23 may take the form of two petals symmetrical about leveling surface 25 at an opening angle (β) between 70° and 85° and connected smoothly to leveling surface 25 having a rounding radius (R₁) between 0.1 and 0.3 mm.

Limiter 23 of fontanel deformation by static load merges with leveling surface 25 to produce a concavity (FIGS. 2 and 3) with a radius (R₂) of 2 to 3 mm and has petals of a rounded profile with a radius (R₃) of 0.3 to 0.5 mm.

Contact surface 24 of limiter 23 of bregmatic fontanel deformation by static load (FIG. 3) has a maximum height (H) of 0.5 to 1.0 mm from the plane tangent to leveling surface 25 of supporting member 3, a length (S) of 2 to 3 mm (FIG. 1), and a width (W) of 4 to 6 mm (FIG. 2), and is formed by curves of radiuses R₄ and R₅ equal to 1.5-2 mm and 4-6 mm, respectively.

Limiter 23 may have other embodiments as well, including an embodiment having a single petal or in the form of one annular projection (not shown in the drawings) having inclined contact surface 24.

Supporting member 3 is secured in the bottom part of movable bush 2 by a resilient clamp 27 (FIG. 1) that allows supporting member 3 to turn around its axis, if needed, or be removed in order for rod 4 to be pulled out for regular cleaning from dirt.

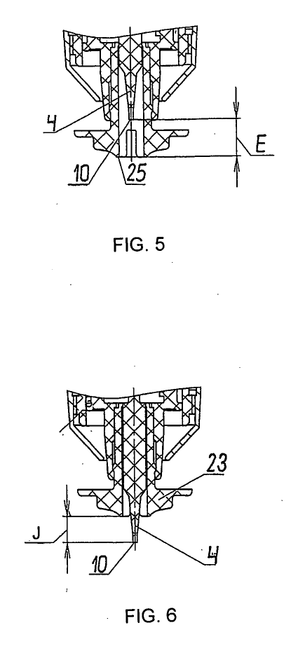

Rod 4 is placed in the cavity of movable bush 2 in its initial position (FIG. 5) such that its flat base 11 is at a distance (E) of 4.5 to 5.0 mm from the plane tangent to leveling surface 25 of the supporting member. The maximum extension of rod 4 out of the cavity of movable bush is equal to a distance (J) of 3.0 to 4.0 mm (FIG. 6).

Body 1 may be housed in a removable protective plastics casing 28 that is rigidly connected to body 1 by a decorative bush 29. In this case, said protective casing 28 and body 1 are adapted to reciprocate vertically together relative to movable bush 2 and its supporting member 3 (FIG. 1) within a range (L) of 4 to 6 mm.

Movable bush 2, fixed coil 19, and supporting member 3 each have respective cavities 30, 31, and 32 and an aperture 33 to release air during the movement of rod 4.

Supporting member 3 has side viewing windows 34 in the form of U-shaped depressions (FIG. 3) or through apertures (FIG. 4) positioned symmetrically between the petals of fontanel deformation limiter 23. Viewing windows 34 have a width (W) of 2 to 3 mm and a height (H) of 2.8 to 3.5 mm, permitting air to escape during the movement of rod 4, giving a view of leveling surface 25 of supporting member 3 when the device is positioned at the center of an open fontanel, and simplifying device positioning on the fontanel.

Supporting member 3 (FIGS. 2, 3, and 4) may have additional widening surfaces 35 of a curvature radius R₆ equal to between 1.5 and 2 mm to stabilize operation of the device in metrological tests.

To better stabilize the position of movable bush 2 and supporting member 3 during ICP measurement, the petals of limiter 23 of fontanel deformation by static load may be provided with recesses 36 (FIG. 4) or through apertures (not shown in the drawings) to produce contact surface 24 along the petal periphery.

In order to simplify the operation of the device, supporting member 3 with limiter 23 of fontanel deformation by static load may be made of a transparent nontoxic material, giving a better view of leveling surface 25 of supporting member 3 when the device is positioned at the center of the bregmatic fontanel.

With the aim of simplifying disinfection and manufacture, supporting member 3 is made integral with limiter 23 of fontanel deformation by static load from a hygienically nontoxic material, for example, medical plastics. Supporting member 3 may be removable and interchangeable for convenience of disinfection.

Control, processing, and display unit 9 serves to switch over, amplify, convert analog to digital signals, have them processed in its microprocessor, and turn on the power source. Said unit 9 switches over signals to reverse the current flow in measuring winding 7 in order to reverse the direction of movement of rod 4 and apply a measured impact force to the fontanel, and is controlled by the microprocessor.

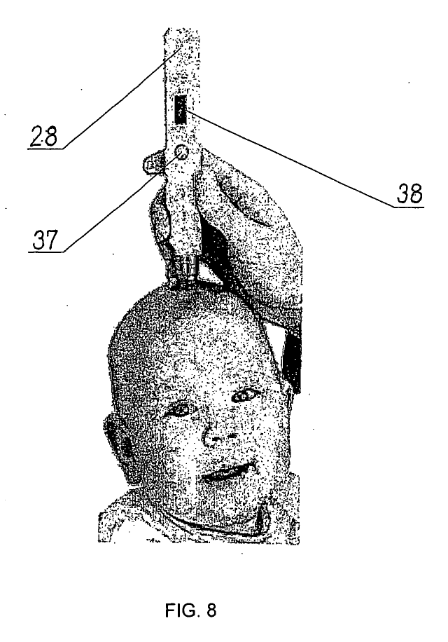

Protective casing 28 (FIG. 8) integrates the device components into a single structure. The face of protective casing 28 has a TURN ON button 37 and a display 38 to represent the ICP measurement results in digital form, which both are elements of control, processing, and display unit 9. The tonometer electric power supply comprises two power storage elements of total 2.4 volts that are placed in a tonometer power supply bay (not shown in the drawings) at the back of protective casing 28.

The operating principle of the device is based on the processing of the movement speed function of rod 4 of a certain weight under the effect of a measured magnetic field and rod interaction with the elastic skin surface in the fontanel area.

The idea of the invention consists in using an impact force to deform a child's bregmatic fontanel by a constant static load that stabilizes the position of the measuring part of the device, and in using the supporting part of the device of a specific form that helps reduce deformation of the fontanel under the effect of said static load. The ICP measuring device applies a measured movement impulse to rod 4 to deform the fontanel and convert the rod speed (as a result of interaction with the elastic surface of the fontanel) to electric current signals.

### PRACTICAL APPLICABILITY

The device is used as follows: Supporting member 3 and the lower part of rod 4 are disinfected. A child is held by its mother or nurse in her hands such that the bregmatic fontanel surface is horizontal. Hair in the area of the child's fontanel is to be combed away from the back of child's head toward its forehead. The child is to be calm or asleep during the ICP measuring procedure. The center of the child's fontanel is located and marked with a blotch of brilliant green 1 to 2 mm in diameter.

The device is held with its support 3 down (FIG. 8), and power is turned on by pushing TURN ON button 37. The device is placed vertically on the child's head, in the area of the fontanel such that the base of tonometer rod 4 is at the center of the fontanel surface area marked with brilliant green. Protective casing 28 of the device is lowered slowly, still in the vertical position, until the characteristic sound of vibration of moving rod 4 is, and will be, heard until the end of the ICP measurement procedure. The device is held in that position immovably (for no more than 1 second) until the sound of vibration of rod 4 ceases, whereupon it is lifted off the fontanel, the digital ICP reading showing on display 38.

Low ICP readings suggest that the area of contact between the fontanel and contact surface 24 of limiter 23 of fontanel deformation by static load be increased to preclude unacceptable deformation of the fontanel.

Deformation limiter 23 allows accurate ICP measurements to be taken owing to guaranteed contact between leveling surface 25 and the fontanel regardless of the shape thereof (flat, concave or convex) because the entire contact surface 24 of deformation limiter 23 is located above leveling surface 25 of supporting member 3.

When protective casing 28 is lowered together with body 1, the movable part of the device (comprising movable bush 2 carrying measuring winding 7, additional bush 18, and supporting member 3) slides upward relative to body 1. As a result, additional bush 18 made, for example, of brass moves up over fixed coil 19 and changes the inductance of winding 20, thereby changing the frequency of generator 22, the winding being connected to the generator circuit by lead 21.

Said generator 22 having attained a certain frequency, control, processing, and display unit 9 feeds d.c. voltage of a specified polarity automatically to measuring winding 7 through lead 8 such that rod 4 is moved up to its initial position by the electromagnetic field of measuring winding 7.

After a while, the d.c. voltage applied previously to measuring winding 7 of coil 6 is cut off automatically, and a short voltage pulse of opposite polarity is applied. As a result, rod 4 is supplied with a measured impulse to start moving toward the fontanel and deform the same with its flat base 10.

As it moves down toward the fontanel, permanent magnet 5 fitted on rod 4 generates a voltage in measuring winding 7 of coil 6, said voltage being then amplified and used for conversion to digital processing and analysis readings of the speed function of rod 4 and shown as ICP measurement results on display 38.

Upper and lower sliding bearings 12 and 11, respectively, allow rod 4 to move freely longitudinally within coil 6 and supporting member 3 during device operation. Downward movement of rod 4 is limited by surface 13 of lower sliding bearing 11. Movement of rod 4 to its top position is limited by surface 14 of the projection of rod 4 and upper sliding bearing 12. Aperture 33 in supporting member 3, cavities 30 and 31 inside movable bush 2, and cavity 32 in fixed coil 19 allow air to escape during movement of rod 4. Mechanical limiter 16 provided in longitudinal slot 15 of body 1 prevents rotation of movable bush 2 around its axis.

A microprocessor that is part of control, processing, and display unit 9 converts the analog signal received from measuring winding 7 to digital form, effects programmed control of the operating modes, digital processing, and movement function analysis of rod 4.

The present device maintains the static load applied to the fontanel stable during measurement (owing to a contactless indicator of required static load and an instruction it generates for measurement to be made automatically), thereby raising the accuracy of ICP measurement. Permanent magnet 5 fitted on rod 4 forces rod 4 to be moved controllably by the electromagnetic field of coil 6 with its measuring winding 7. This design lowers significantly the requirement that the device be held vertically during measurement and allows rod 4 to return to its initial position automatically and several ICP measurements to be made successively at the same device position on the fontanel.

The ICP measuring device is convenient because all the elements thereof are enclosed in the same casing measuring 174×26×20 mm at most and weighing not more than 100 grams.

The present ICP measuring device has an informative accuracy of static load setting during ICP measurement, making measurement accurate and fast. The measurement procedure is painless. ICP is measured in millimeters of mercury column with an accuracy of ±0.4 mm of mercury column. Measurement does not take more than 1 second.

Taking many ICP measurements in one child in 24 hours at a minimum cost in time is very important for verifying the choice of treatment method and raises its efficiency significantly. There is no risk of infection during measurement because the device operates out of direct contact with the brain.

Simple design allows the device to be manufactured at affordable prices, and its simple operation allows it to be used in clinics and at home. The present device helps improve the accuracy of ICP measurement and allows changes in ICP to be monitored during treatment.

The device offers the following advantages:
noninvasive ICP measurement;
painless ICP measurement;
high accuracy of ICP measurement on fontanels of different shapes, including concave, flat, and convex shapes;
reduction in total time required for an ICP measurement procedure;
reduction in the time needed to manipulate the device into an operating position;
application in any conditions, requiring no specialized personnel training;
improved consumer properties of the device (comfort, convenient use, and stable parameters); and
simple operation of the device.

The present device is convenient and reliable in operation, simple and inexpensive to manufacture, safe and suitable for a variety of purposes, does not require straps or adhesive to be kept in place, has a long service life, and can be disinfected repeatedly to comply with sanitary and hygienic standards.

What is claimed is:

1. A device for measuring intracranial pressure in newborns and babies, comprising a body, a supporting member having a leveling surface at the end thereof, a movable bush with said supporting member secured coaxially thereon, said movable bush being disposed in the body for limited reciprocation to apply static load to the bregmatic fontanel, a rod having a flat base and disposed in the cavity of the movable bush for movement relative to the supporting member and producing an impact force to deform the bregmatic fontanel, a system causing said rod to move, said system having at least one measuring winding connected to a control, processing, and display unit adapted to convert a signal it receives from the measuring winding to determine intracranial pressure and display the results obtained, wherein the supporting member has on the outer surface thereof a limiter of bregmatic fontanel deformation by static load, the leveling surface of the supporting member extending beyond the contact surface of said limiter, said contact surface being inclined to a plane normal to the axis of the movable bush and tangent to the leveling surface of the supporting member with increasing distance therefrom to said plane in the direction toward the periphery of the limiter of bregmatic fontanel deformation by static load.

2. The device as claimed in claim 1, wherein the limiter of bregmatic fontanel deformation by static load is at least one projection provided on the side surface of the supporting member, the inclination angle of the contact surface thereof to said plane tangent to the leveling surface varying from 5° to 15°.

3. The device as claimed in claim 1, wherein the distance from the plane tangent to the leveling surface to a point furthest therefrom on the contact surface of the limiter of bregmatic fontanel deformation by static load ranges from 0.5 to 1.0 mm.

4. The device as claimed in claim 1, wherein the limiter of bregmatic fontanel deformation by static load is a pair of symmetrical petals, the contact surface of each of them merging smoothly with the leveling surface of the supporting member.

5. The device as claimed in claim 4, wherein each petal of the limiter of bregmatic fontanel deformation by static load has an opening angle of 70° to 85°, a length of 2 to 3 mm along the common axis of symmetry of the petals, and a width of 4 to 6 mm.

6. The device as claimed in claim 1, wherein the movable bush and the supporting member have respective cavities and apertures to release air during the movement of the rod.

7. The device as claimed in claim 4, wherein the supporting member has side viewing windows in the form of U-shaped depressions or apertures located opposite one another between the petals.

8. The device as claimed in claim 1, wherein the leveling surface of the supporting member has the shape of a continuous or split ring and is rounded in the axial section thereof.

9. The device as claimed in claim 1, wherein the rod has a flat base between 0.2 and 1.5 mm² in area.

10. The device as claimed in claim 1, wherein the supporting member is a removable bush.

11. The device as claimed in claim 1, wherein the system causing the rod to move is of the electromagnetic type and comprises a permanent magnet disposed on the rod, a measuring winding disposed in the movable bush and positioned with respect to the permanent magnet to control the direction and movement speed of the rod.

12. The device as claimed in claim 1, wherein the control, processing, and display unit is adapted to produce voltage of opposite polarities across the measuring winding to retract the rod to the initial position thereof and produce thereafter an impulse to cause the rod to move in the direction of the supporting member.

13. The device as claimed in claim 1 further provided with a contactless indicator of a reference static load applied to the bregmatic fontanel during measurement, said indicator being a sensor indicating the position of the bush relative to the body.

14. The device as claimed in claim 13, wherein the sensor indicating the position of the movable bush relative to the body comprises a generator, an additional bush with a cavity, and a coil disposed on the movable bush and the body, respectively, to vary the inductance of the coil winding in response to the movement of the movable bush, said coil winding being connected to the generator circuit.

15. The device as claimed in claim 14, wherein the generator of the sensor indicating the position of the movable bush relative to the body is connected to the control, processing, and measurement data display unit.

16. A supporting member for a device for measuring intracranial pressure in newborns and babies in the form of a bush having a leveling surface at the end thereof, said supporting member further having on the outer surface thereof a limiter of bregmatic fontanel deformation by static load, the leveling surface thereof extending beyond the contact surface of said limiter, the contact surface being inclined to a plane tangent to the leveling surface, with increasing distance therefrom to said plane in the direction toward the periphery of the limiter of bregmatic fontanel deformation by static load.

17. A supporting member as claimed in claim 16, wherein the limiter of bregmatic fontanel deformation by static load is at least one projection on the side surface thereof having a contact surface inclined to said plane tangent to the leveling surface at an angle of 5° to 15°.

18. A supporting member as claimed in claim 16, wherein the distance from said plane tangent to the leveling surface to a point furthest therefrom on the contact surface of the limiter of bregmatic fontanel deformation by static load ranges from 0.5 to 1.0 mm.

19. A supporting member as claimed in claim 16, wherein the limiter of bregmatic fontanel deformation by static load is formed by a pair of symmetrical petals each having a contact surface merging smoothly with the leveling surface.

20. A supporting member as claimed in claim 19, wherein each petal of the limiter of bregmatic fontanel deformation by static load has an opening angle of 70° to 85°, a length of 2 to 3 mm along the common axis of symmetry of the petals, and a width of 4 to 6 mm.

21. A supporting member as claimed in claim 19 further having side viewing windows in the form of U-shaped depressions or apertures disposed opposite one another between the petals.

22. A supporting member as claimed in claim 16, wherein the leveling surface thereof is rounded in the axial section thereof and has the form of a continuous or split ring.

23. A supporting member as claimed in claim 16 further having a bore for the rod of the intracranial pressure measuring device to move therein and apertures to release air during the movement of said rod.
